(19) United States
(12) Patent Application Publication
(10) Pub. No.: US 2022/0234921 A1
(43) Pub. Date: Jul. 28, 2022

(54) DISINFECTION SYSTEM CAPABLE OF DIRECTLY GENERATING OZONE WATER IN WATER PIPE WITH ON-OFF CONTROL DEVICE

(71) Applicant: Yantai United Ozonetec Corporation, Yantai (CN)

(72) Inventors: MING-YUNG HSU, Yantai (CN); WEI-CHUN HSU, Yantai (CN)

(21) Appl. No.: 17/161,634

(22) Filed: Jan. 28, 2021

(51) Int. Cl. C02F 1/78

(52) U.S. Cl. CPC C02F 1/78; C02F 2201/005; C02F 2303/04; C02F 2209/40; C02F 2209/23; C02F 2201/782

(57) ABSTRACT

A disinfection system includes an ozone water generator and a top seat movably connected to the ozone water generator. The top seat includes a generator control board and an on-off control device therein. The on-off control device includes a flow switch. The flow switch is connected to the generator control board through a signal wire. A top seat inlet of the top seat is in communication with a top seat outlet of the top seat through an inlet of the flow switch, an outlet of the flow switch, a generator inlet of the ozone water generator, a generator outlet of the ozone water generator and a pipe. The ozone water generator is connected to the generator control board through a connecting wire. The generator control board is connected to an external power source through a power wire. The disinfection system can sense changes in water flow and directly generate ozone water according to needs, thereby improving the convenience for use, ensuring the sensitivity of the flow switch and increasing the maximum water flow range.

### FIELD OF THE INVENTION

The present invention relates to a disinfection system, and more particularly to a disinfection system capable of directly generating ozone water in a water pipe with an on-off control device.

### BACKGROUND OF THE INVENTION

A water pipe may gradually produce bacteria on the wall of the pipe during use. Ozone water has the advantages of efficient sterilization and no residue, so it is widely used for sterilization. Ozone water is generally produced by mixing ozone gas and water. Ozone gas is generally produced by a high-voltage discharge method and low-voltage electrolysis method. The low-voltage electrolysis method uses an ozone water generator that electrolyzes water to produce ozone, and its anode material is lead dioxide. Because lead dioxide is poison and the catalytic activity is reduced, tap water cannot be used directly as water for electrolysis. It is necessary to electrolyze pure water to generate ozone gas and then mix with water. Therefore, ozone water equipment needs a pure water device and a mixing device, which increases the complexity of the equipment, increases the volume of the equipment and increases the cost of the equipment.

Accordingly, the inventor of the present invention has devoted himself based on his many years of practical experiences to solve these problems.

### SUMMARY OF THE INVENTION

The primary objective of the present invention is to overcome the above-mentioned shortcomings of the prior art and to provide a disinfection system capable of directly generating ozone water in a water pipe with an on-off control device.

In order to achieve the above object, a disinfection system is provided. The disinfection system comprises an ozone water generator and a top seat movably connected to the ozone water generator. The top seat includes a generator control board and an on-off control device therein. The on-off control device includes a flow switch. The flow switch is connected to the generator control board through a signal wire. A top seat inlet of the top seat is in communication with a top seat outlet of the top seat through an inlet of the flow switch, an outlet of the flow switch, a generator inlet of the ozone water generator, a generator outlet of the ozone water generator and a pipe. The ozone water generator is connected to the generator control board through a connecting wire. The generator control board is connected to an external power source through a power wire.

Preferably, the top seat inlet is connected to the inlet of the flow switch through the pipe. The outlet of the flow switch is connected to the generator inlet of the ozone water generator through the pipe. The generator outlet of the ozone water generator is connected to the top seat outlet of the top seat through the pipe.

Preferably, the on-off control device further includes a damping valve. An inlet of the damping valve is connected to the top seat inlet of the top seat through the pipe. An outlet of the damping valve is connected to the top seat outlet of the top seat through the pipe.

Alternatively, the on-off control device further includes a damping valve. The top seat inlet of the top seat is connected to the inlet of the flow switch and an inlet of the damping valve through the pipe. An outlet of the damping valve is connected to the generator inlet of the ozone water generator through the pipe. The generator outlet of the ozone water generator and an outlet of the flow switch are connected to the top seat outlet of the top seat through the pipe.

The beneficial effects of the present invention are described below. The on-off control device includes a flow switch to sense the change of the water flow and directly generate ozone water as needed, thereby improving convenience for use. The on-off control device further includes a damping valve, thereby ensuring the sensitivity of the flow switch and increasing the maximum water flow range.

### BRIEF DESCRIPTION OF THE DRAWINGS

FIG. 1 is a structural schematic view of a first embodiment of the present invention;

FIG. 2 is a structural schematic view of a second embodiment of the present invention; and

FIG. 3 is a structural schematic view of a second embodiment of the present invention.

### DETAILED DESCRIPTION OF THE PREFERRED EMBODIMENTS

Embodiments of the present invention will now be described, by way of example only, with reference to the accompanying drawings.

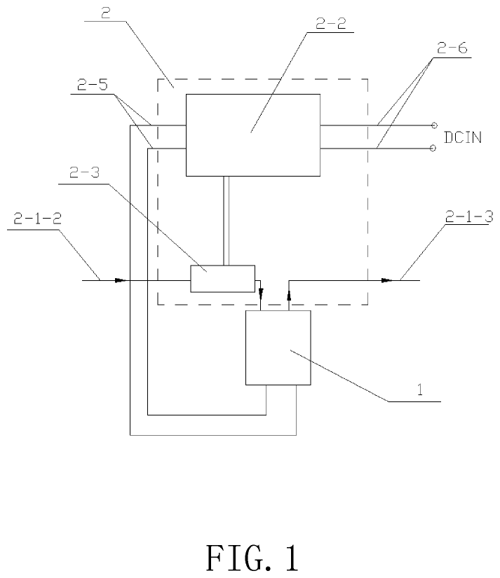

FIG. 1 illustrates a first embodiment of a disinfection system capable of directly generating ozone water in a water pipe with an on-off control device. The disinfection system comprises an ozone water generator 1 and a top seat 2 movably connected to the ozone water generator 1. The top seat 2 includes a generator control board 2-2 and an on-off control device therein. The on-off control device includes a flow switch 2-3. The flow switch 2-3 is connected to the generator control board 2-2 through a signal wire. A top seat inlet 2-1-2 of the top seat 2 is connected to an inlet of the flow switch 2-3 through a pipe. An outlet of the flow switch 2-3 is connected to a generator inlet of the ozone water generator 1 through a pipe. A generator outlet of the ozone water generator 1 is connected to a top seat outlet 2-1-3 of the top seat 2 through a pipe. The ozone water generator 1 is connected to the generator control board 2-3 through a connecting wire 2-5. The generator control board 2-3 is connected to an external power source through a power wire 2-6.

When in use, the disinfection system is powered and connected with a water source. When there is water flowing in the pipe, water with conductivity greater than 30 μs/cm will flow through the top seat inlet 2-1-2 and the generator inlet to enter the generator. The signal of the flow switch 2-3 is transmitted to the generator control board 2-2. The generator control board 2-2 supplies a constant current power supply to the generator through the connecting wire. The voltage range of the power supply is 3.5-12 V to start the generator. Water is electrolyzed under the action of an electric field, and oxygen ions generate ozone and oxygen microbubbles under the action of the anode catalyst. Ozone microbubbles quickly dissolve into water to directly generate ozone water. The ozone water flows out through the generator outlet and the top seat outlet 2-1-3 for removing the biofilm on the pipe wall and sterilizing the water in the pipe. When the water in the pipe stops flowing, the generator control board 2-2 gets the signal that the water stops flowing to immediately stop powering the ozone water generator 1. If the user wants to get a higher concentration of ozone water for next use, the ozone water generator 1 continues to be powered for a period of time by setting the generator control board 2-2 before stopping the power supply, for example, an optimal delay of 10 seconds. In this way, the high-concentration ozone water is stored in the ozone water generator 1, and the high-concentration ozone water will be provided instantaneously in the next use, so as to meet some application fields.

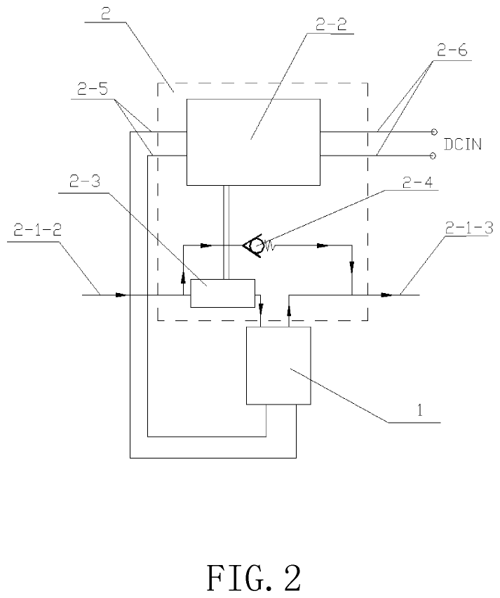

FIG. 2 illustrates a second embodiment of a disinfection system capable of directly generating ozone water in a water pipe with an on-off control device. The disinfection system comprises an ozone water generator 1 and a top seat 2 movably connected to the ozone water generator 1. The top seat 2 includes a generator control board 2-2 and an on-off control device therein. The on-off control device includes a flow switch 2-3 and a damping valve 2-4. The flow switch 2-3 is connected to the generator control board 2-2 through a signal wire. A top seat inlet 2-1-2 of the top seat 2 is connected to an inlet of the flow switch 2-3 and an inlet of the damping valve 2-4 through a pipe. An outlet of the flow switch 2-3 is connected to a generator inlet of the ozone water generator 1 through a pipe. A generator outlet of the ozone water generator 1 and an outlet of the damping valve 2-4 are connected to a top seat outlet 2-1-3 of the top seat 2 through a pipe. The ozone water generator 1 is connected to the generator control board 2-3 through a connecting wire 2-5. The generator control board 2-3 is connected to an external power source through a power wire 2-6.

When in use, the disinfection system is powered and connected with a water source. When there is water flowing in the pipe, water with conductivity greater than 30 μs/cm will flow through the top seat inlet 2-1-2 to be divided into two ways, one flowing through the damping valve 2-2 to the top seat outlet 2-1-3 and the other flowing through the flow switch 2-3 and the generator inlet to enter the generator. The function of the damping valve 2-2 is to ensure that the flow switch 2-3 gets enough water flow, enabling the flow switch 2-3 to be more sensitive. The signal of the flow switch 2-3 is transmitted to the generator control board 2-2. The generator control board 2-2 supplies a constant current power supply to the generator through the connecting wire, so as to start the generator. Water is electrolyzed under the action of an electric field, and oxygen ions generate ozone and oxygen microbubbles under the action of the anode catalyst. Ozone microbubbles quickly dissolve into water to directly generate ozone water. The ozone water flowing through the generator outlet and the water flowing through the damping valve are mixed to flow out through the top seat outlet 2-1-3 for removing the biofilm on the pipe wall and sterilizing the water in the pipe. When the water in the pipe stops flowing, the generator control board 2-2 gets the signal that the water in the pipe stops flowing to immediately stop powering the ozone water generator 1. If the user wants to get a higher concentration of ozone water for next use, the ozone water generator 1 continues to be powered for a period of time by setting the generator control board 2-2 before stopping the power supply, for example, an optimal delay of 10 seconds. In this way, the high-concentration ozone water is stored in the ozone water generator 1, and the high-concentration ozone water will be provided instantaneously in the next use, so as to meet some application fields.

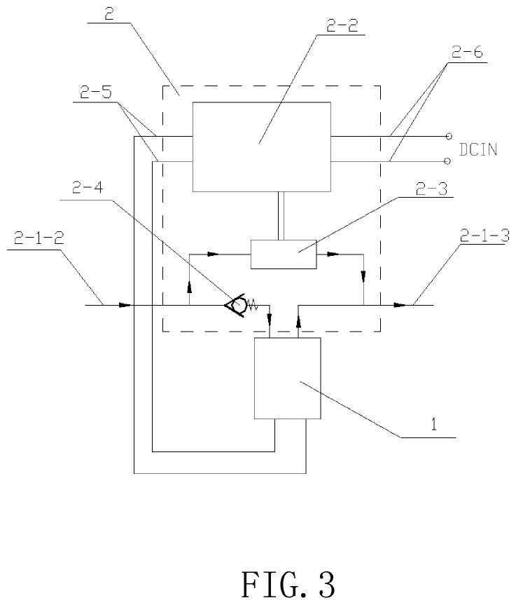

FIG. 3 illustrates a third embodiment of a disinfection system capable of directly generating ozone water in a water pipe with an on-off control device. The disinfection system comprises an ozone water generator 1 and a top seat 2 movably connected to the ozone water generator 1. The top seat 2 includes a generator control board 2-2 and an on-off control device therein. The on-off control device includes a flow switch 2-3 and a damping valve 2-4. The flow switch 2-3 is connected to the generator control board 2-2 through a signal wire. A top seat inlet 2-1-2 of the top seat 2 is connected to an inlet of the flow switch 2-3 and an inlet of the damping valve 2-4 through a pipe. An outlet of the damping valve 2-4 is connected to a generator inlet of the ozone water generator 1 through a pipe. A generator outlet of the ozone water generator 1 and an outlet of the flow switch 2-3 are connected to a top seat outlet 2-1-3 of the top seat 2 through a pipe. The ozone water generator 1 is connected to the generator control board 2-3 through a connecting wire 2-5. The generator control board 2-3 is connected to an external power source through a power wire 2-6.

When in use, the disinfection system is powered and connected with a water source. When there is water flowing in the pipe, water with conductivity greater than 30 μs/cm will flow through the top seat inlet 2-1-2 to be divided into two ways, one flowing through the flow switch 2-3 to the top seat outlet 2-1-3 and the other flowing through the damping valve 2-4 and the generator inlet to enter the generator. The function of the damping valve 2-2 is to ensure that the flow switch 2-3 gets enough water flow, enabling the flow switch 2-3 to be more sensitive. The signal of the flow switch 2-3 is transmitted to the generator control board 2-2. The generator control board 2-2 supplies a constant current power supply to the generator through the connecting wire, so as to start the generator. Water is electrolyzed under the action of an electric field, and oxygen ions generate ozone and oxygen microbubbles under the action of the anode catalyst. Ozone microbubbles quickly dissolve into water to directly generate ozone water. The ozone water flowing through the generator outlet and the water flowing through the flow switch are mixed to flow out through the top seat outlet 2-1-3 for removing the biofilm on the pipe wall and sterilizing the water in the pipe. When the water in the pipe stops flowing, the generator control board 2-2 gets the signal that the water in the pipe stops flowing to immediately stop powering the ozone water generator 1. If the user wants to get a higher concentration of ozone water for next use, the ozone water generator 1 continues to be powered for a period of time by setting the generator control board 2-2 before stopping the power supply, for example, an optimal delay of 10 seconds. In this way, the high-concentration ozone water is stored in the ozone water generator 1, and the high-concentration ozone water will be provided instantaneously in the next use, so as to meet some application fields.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A disinfection system, comprising an ozone water generator and a top seat movably connected to the ozone water generator, the top seat including a generator control board and an on-off control device therein, the on-off control device including a flow switch, the flow switch being connected to the generator control board through a signal wire, a top seat inlet of the top seat being in communication with a top seat outlet of the top seat through an inlet of the flow switch, an outlet of the flow switch, a generator inlet of the ozone water generator, a generator outlet of the ozone water generator and a pipe; the ozone water generator being connected to the generator control board through a connecting wire, the generator control board being connected to an external power source through a power wire.

2. The disinfection system as claimed in claim 1, wherein the top seat inlet is connected to the inlet of the flow switch through the pipe, the outlet of the flow switch is connected to the generator inlet of the ozone water generator through the pipe, and the generator outlet of the ozone water generator is connected to the top seat outlet of the top seat through the pipe.

3. The disinfection system as claimed in claim 2, wherein the on-off control device further includes a damping valve, an inlet of the damping valve is connected to the top seat inlet of the top seat through the pipe, and an outlet of the damping valve is connected to the top seat outlet of the top seat through the pipe.

4. The disinfection system as claimed in claim 1, wherein the on-off control device further includes a damping valve, the top seat inlet of the top seat is connected to the inlet of the flow switch and an inlet of the damping valve through the pipe, an outlet of the damping valve is connected to the generator inlet of the ozone water generator through the pipe, the generator outlet of the ozone water generator and an outlet of the flow switch are connected to the top seat outlet of the top seat through the pipe.
